(19) United States
(12) Patent Application Publication
(10) Pub. No.: US 2026/0142028 A1
(43) Pub. Date: May 21, 2026

(54) SYSTEMS AND METHOD FOR MEDICAL DATA MANAGEMENT

(71) Applicant: Express Scripts Strategic Development, Inc., St. Louis, MO (US)

(72) Inventors: Rebecca Lyss, St. Louis, MO (US); Jacqueline Luong, Land O Lakes, FL (US); Navathej Lingopalli, Plano, TX (US); Dani Felder, St. Louis, MO (US); Brittney Williams, St. Louis, MO (US); Akhila Thadikonda, St. Louis, MO (US); Anders Ravenholt, St. Louis, MO (US); Seetharaman Srinivasan, St. Louis, MO (US)

(21) Appl. No.: 19/391,733

(22) Filed: Nov. 17, 2025

Related U.S. Application Data
(60) Provisional application No. 63/720,879, filed on Nov. 15, 2024.
(60) Provisional application No. 63/728,723, filed on Dec. 6, 2024.

(51) Int. Cl. G16H 40/67; G16H 10/60; G16H 40/20

(52) U.S. Cl. CPC G16H 40/67; G16H 10/60; G16H 40/20

(57) ABSTRACT

An example computer system includes memory hardware and processor hardware configured to execute instructions including receiving a medical appointment slot swap request from a first patient, accessing a set of swappable medical appointment slots from a database, receiving a selection of one of the set of swappable medical appointment slots from the first patient, transmitting a notification to a user interface corresponding to a second patient associated with the selected one of the set of swappable medical appointment slots, receiving man approval decision input from the second patient, and in response to receiving the swap acceptance, cancelling a current booked status of the selected one of the set of swappable medical appointment slots associated with the second patient, and assigning a booked status of the selected one of the set of swappable medical appointment slots to the first patient.

### CROSS-REFERENCE TO RELATED APPLICATIONS

This application claims the benefit and priority of U.S. Provisional Application No. 63/720,879, filed on Nov. 15, 2024, and U.S. Application No. 63/728,723, filed on Dec. 6, 2024. The entire disclosures of each of the above applications are incorporated herein by reference.

### FIELD

The present disclosure relates to systems and methods for medical data management, e.g., provider data management, appointment swap management data and the like.

### BACKGROUND

Patients often book medical appointments in advance, based on schedules of available appointments for different physicians. Limited schedule availability for physicians may prevent patients from accessing desired appointment time slots.

The background description provided here is for the purpose of generally presenting the context of the disclosure. Work of the presently named inventors, to the extent it is described in this background section, as well as aspects of the description that may not otherwise qualify as prior art at the time of filing, are neither expressly nor impliedly admitted as prior art against the present disclosure.

### SUMMARY

An example computer system includes memory hardware configured to store medical appointment data, appointment swap preference data, and computer-executable instructions, and processor hardware configured to execute the instructions. The instructions include receiving, via a first user interface, a medical appointment slot swap request from a first patient, wherein the first user interface is a user interface of a first user device remote from a database of the memory hardware, accessing a set of swappable medical appointment slots from the database of the memory hardware, wherein each of the set of swappable medical appointment slots comprises a medical appointment slot having an availability status set to booked in the medical appointment data, and a swap possible status set to true in the appointment swap preference data, and accessing the set of swappable medical appointment slots includes identifying a storage location in the database of the memory hardware and executing a read instruction to read electronically stored data from the database at the identified storage location, electronically transmitting the set of swappable medical appointment slots to the first user device via at least one of a wired communication network and a wireless communication network, receiving, via the first user interface, a selection of one of the set of swappable medical appointment slots from the first patient, transmitting a notification to a second user interface corresponding to a second patient associated with the selected one of the set of swappable medical appointment slots, receiving, via the second user interface, an approval decision input from the second patient, wherein the approval decision input includes a swap acceptance or a swap denial, and the second user interface is a user interface of a second user device remote from the database of the memory hardware, and in response to receiving the swap acceptance, cancelling a current booked status of the selected one of the set of swappable medical appointment slots associated with the second patient, and assigning a booked status of the selected one of the set of swappable medical appointment slots to the first patient, wherein cancelling the current booked status and assigning the booked status includes modifying at least one electronically stored data entry of the database of the memory hardware to electronically store modified data.

In some examples, the processor hardware is configured to execute the instructions to, in response to receiving the swap acceptance, identify an alternative medical appointment slot having an availability status set to available, and assign a booked status of the alternative medical appointment slot to the second patient.

In some examples, the processor hardware is configured to execute the instructions to, in response to receiving the swap denial, transmit a notification to the first user interface indicative of the swap denial.

In some examples, the processor hardware is configured to execute the instructions to, in response to receiving the swap denial, identify an alternative medical appointment slot having an availability status set to available, and assign a booked status of the alternative medical appointment slot to the first patient.

In some examples, the processor hardware is configured to execute the instructions to, in response to receiving the selection of one of the set of swappable medical appointment slots from the first patient, add the selected one of the set of swappable medical appointment slots to a waitlist, and assign a high priority value and swap request appointment description to the selected one of the set of swappable medical appointment slots.

In some examples, adding the selected one of the set of swappable medical appointment slots to a waitlist includes setting a request status value associated with the selected one of the set of swappable medical appointment slots to waitlist, and setting a practitioner status value associated with the selected one of the set of swappable medical appointment slots to action needed.

In some examples, assigning the booked status includes storing appointment details in the database via an appointment entity application programming interface (API).

In some examples, the processor hardware is configured to execute the instructions to receive a cancellation of a medical appointment slot having an availability status set to booked, and transmit a notification to the first user interface in response to the first patient having a waitlist request for the medical appointment slot, wherein the notification indicates availability of the medical appointment slot.

In some examples, the processor hardware is configured to execute the instructions to display an acceptance option on the first user interface, receive an acceptance option input selection from the first patient via the first user interface, and in response to the acceptance option input selection including a slot acceptance, assign a booked status of the medical appointment slot to the first patient.

In some examples, the processor hardware is configured to execute the instructions to, in response to the acceptance option input selection including a slot denial, cancel the waitlist request for the medical appointment slot associated with the first patient.

An example method for medical appointment swap management includes storing medical appointment data and appointment swap preference data in memory hardware, receiving, via a first user interface, a medical appointment slot swap request from a first patient, wherein the first user interface is a user interface of a first user device remote from a database of the memory hardware, accessing a set of swappable medical appointment slots from a database of the memory hardware, wherein each of the set of swappable medical appointment slots comprises a medical appointment slot having an availability status set to booked in the medical appointment data, and a swap possible status set to true in the appointment swap preference data, and accessing the set of swappable medical appointment slots includes identifying a storage location in the database of the memory hardware and executing a read instruction to read electronically stored data from the database at the identified storage location, electronically transmitting the set of swappable medical appointment slots to the first user device via at least one of a wired communication network and a wireless communication network, receiving, via the first user interface, a selection of one of the set of swappable medical appointment slots from the first patient, transmitting a notification to a second user interface corresponding to a second patient associated with the selected one of the set of swappable medical appointment slots, receiving, via the second user interface, an approval decision input from the second patient, wherein the approval decision input includes a swap acceptance or a swap denial, and the second user interface is a user interface of a second user device remote from the database of the memory hardware, and in response to receiving the swap acceptance, cancelling a current booked status of the selected one of the set of swappable medical appointment slots associated with the second patient, and assigning a booked status of the selected one of the set of swappable medical appointment slots to the first patient, wherein cancelling the current booked status and assigning the booked status includes modifying at least one electronically stored data entry of the database of the memory hardware to electronically store modified data.

In some examples, the method includes, in response to receiving the swap acceptance identifying an alternative medical appointment slot having an availability status set to available, and assigning a booked status of the alternative medical appointment slot to the second patient.

In some examples, the method includes, in response to receiving the swap denial, transmitting a notification to the first user interface indicative of the swap denial.

In some examples, the method includes, in response to receiving the swap denial, identifying an alternative medical appointment slot having an availability status set to available, and assigning a booked status of the alternative medical appointment slot to the first patient.

In some examples, the method includes, in response to receiving the selection of one of the set of swappable medical appointment slots from the first patient, adding the selected one of the set of swappable medical appointment slots to a waitlist, and assigning a high priority value and swap request appointment description to the selected one of the set of swappable medical appointment slots.

In some examples, adding the selected one of the set of swappable medical appointment slots to a waitlist includes setting a request status value associated with the selected one of the set of swappable medical appointment slots to waitlist, and setting a practitioner status value associated with the selected one of the set of swappable medical appointment slots to action needed.

In some examples, assigning the booked status includes storing appointment details in the database via an appointment entity application programming interface (API).

In some examples, the method includes receiving a cancellation of a medical appointment slot having an availability status set to booked, and transmitting a notification to the first user interface in response to the first patient having a waitlist request for the medical appointment slot, wherein the notification indicates availability of the medical appointment slot.

In some examples, the method includes displaying an acceptance option on the first user interface, receiving an acceptance option input selection from the first patient via the first user interface, and in response to the acceptance option input selection including a slot acceptance, assigning a booked status of the medical appointment slot to the first patient.

In some examples, the method includes, in response to the acceptance option input selection including a slot denial, cancelling the waitlist request for the medical appointment slot associated with the first patient.

Further areas of applicability of the present disclosure will become apparent from the detailed description, the claims, and the drawings. The detailed description and specific examples are intended for purposes of illustration only and are not intended to limit the scope of the disclosure.

### BRIEF DESCRIPTION OF THE DRAWINGS

The present disclosure will become more fully understood from the detailed description and the accompanying drawings.

FIG. 1 is a functional block diagram of an example system including a high-volume pharmacy.

FIG. 2 is a functional block diagram of an example pharmacy fulfillment device, which may be deployed within the system of FIG. 1.

FIG. 3 is a functional block diagram of an example order processing device, which may be deployed within the system of FIG. 1.

FIG. 4 is a functional block diagram of an example system for medical appointment swap management.

FIG. 5 is a message sequence chart illustrating example interactions between components of the system of FIG. 4.

FIG. 6 is a flowchart depicting an example process for requesting a medical appointment using an interface with a medical appointment scheduling system.

FIG. 7 is a flowchart depicting an example process for booking a desired available appointment slot.

FIG. 8 is a flowchart depicting an example process for adding a request for an unavailable appointment slot to a waitlist.

FIG. 9 is a flowchart depicting an example process for revolving waitlisted appointment requests in response to a desired appointment slot becoming available.

FIG. 10 is a flowchart depicting an example process for checking a status of swap willingness for a patient with a booked appointment.

FIG. 11 is a flowchart depicting an example process for swapping a booked appointment slot in response to a swap request from a patient.

FIG. 12 is a flowchart depicting an example process for modifying a status of opt-in and opt-out swap preferences for a patient.

FIG. 13 is a block diagram of an example generative artificial intelligence system for use with medical appointment swap management.

FIG. 14 is a graphical representation of layers or an example long short-term memory (LSTM) machine learning model.

In the drawings, reference numbers may be reused to identify similar and/or identical elements.

### DETAILED DESCRIPTION

### High-Volume Pharmacy

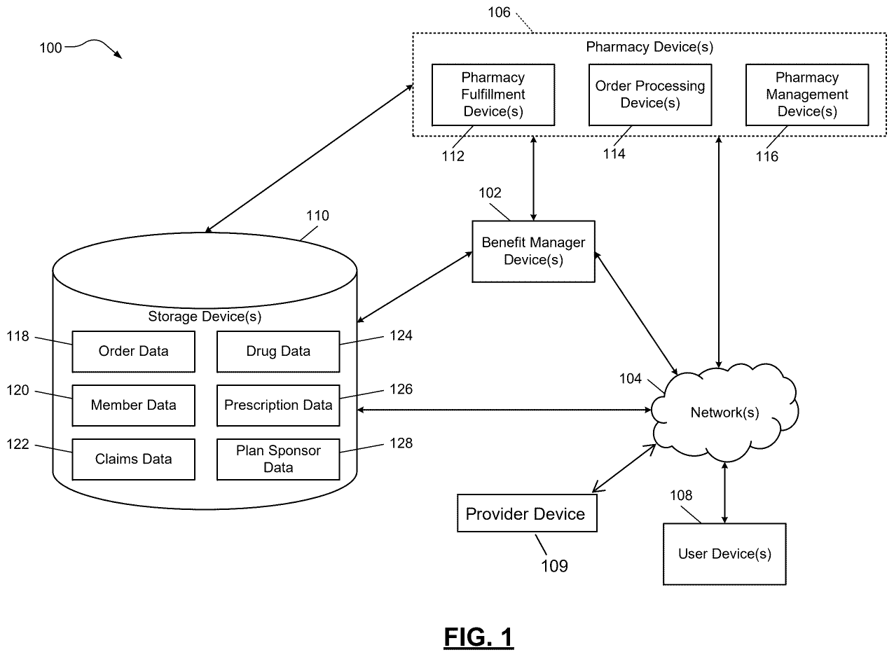

FIG. 1 is a block diagram of an example implementation of a system 100 for a high-volume pharmacy. While the system 100 is generally described as being deployed in a high-volume pharmacy or a fulfillment center (for example, a mail order pharmacy, a direct delivery pharmacy, etc.), the system 100 and/or components of the system 100 may otherwise be deployed (for example, in a lower-volume pharmacy, etc.). A high-volume pharmacy may be a pharmacy that is capable of filling at least some prescriptions mechanically. The system 100 may include a benefit manager device 102 and a pharmacy device 106 in communication with each other directly and/or over a network 104.

The system 100 may also include one or more user device(s) 108. A user, such as a pharmacist, patient, data analyst, health plan administrator, etc., may access the benefit manager device 102 or the pharmacy device 106 using the user device 108. The user device 108 may be a desktop computer, a laptop computer, a tablet, a smartphone, etc.

The benefit manager device 102 is a device operated by an entity that is at least partially responsible for creation and/or management of the pharmacy or drug benefit. While the entity operating the benefit manager device 102 is typically a pharmacy benefit manager (PBM), other entities may operate the benefit manager device 102 on behalf of themselves or other entities (such as PBMs). For example, the benefit manager device 102 may be operated by a health plan, a retail pharmacy chain, a drug wholesaler, a data analytics or other type of software-related company, etc. In some implementations, a PBM that provides the pharmacy benefit may provide one or more additional benefits including a medical or health benefit, a dental benefit, a vision benefit, a wellness benefit, a radiology benefit, a pet care benefit, an insurance benefit, a long term care benefit, a nursing home benefit, etc. The PBM may, in addition to its PBM operations, operate one or more pharmacies. The pharmacies may be retail pharmacies, mail order pharmacies, etc.

Some of the operations of the PBM that operates the benefit manager device 102 may include the following activities and processes. A member (or a person on behalf of the member) of a pharmacy benefit plan may obtain a prescription drug at a retail pharmacy location (e.g., a location of a physical store) from a pharmacist or a pharmacist technician. The member may also obtain the prescription drug through mail order drug delivery from a mail order pharmacy location, such as the system 100. In some implementations, the member may obtain the prescription drug directly or indirectly through the use of a machine, such as a kiosk, a vending unit, a mobile electronic device, or a different type of mechanical device, electrical device, electronic communication device, and/or computing device. Such a machine may be filled with the prescription drug in prescription packaging, which may include multiple prescription components, by the system 100. The pharmacy benefit plan is administered by or through the benefit manager device 102.

In various embodiments, the benefit manager device 102 can include memory hardware (e.g., storage device 110) configured to store medical appointment data, appointment swap preference data, and computer-executable instructions and processor hardware configured to execute the instructions. The instructions can include receiving, via a first user interface, a medical appointment slot swap request from a first patient; accessing a set of swappable medical appointment slots from a database of the memory hardware, wherein each of the set of swappable medical appointment slots comprises a medical appointment slot having an availability status set to booked in the medical appointment data, and a swap possible status set to true in the appointment swap preference data. The instructions can further include receiving, via the first user interface, a selection of one of the set of swappable medical appointment slots from the first patient. The instructions can further include transmitting a notification to a second user interface corresponding to a second patient associated with the selected one of the set of swappable medical appointment slots. The instructions can further include receiving, via the second user interface, an approval decision input from the second patient, wherein the approval decision input includes a swap acceptance or a swap denial. The instructions can further include, in response to receiving the swap acceptance, cancelling a current booked status of the selected one of the set of swappable medical appointment slots associated with the second patient, and assigning a booked status of the selected one of the set of swappable medical appointment slots to the first patient. This may provide more efficient medical care and reduce the use of electronic resources in scheduling and rescheduling medical care.

The member may have a copayment for the prescription drug that reflects an amount of money that the member is responsible to pay the pharmacy for the prescription drug. The money paid by the member to the pharmacy may come from, as examples, personal funds of the member, a health savings account (HSA) of the member or the member's family, a health reimbursement arrangement (HRA) of the member or the member's family, or a flexible spending account (FSA) of the member or the member's family. In some instances, an employer of the member may directly or indirectly fund or reimburse the member for the copayments.

The amount of the copayment required by the member may vary across different pharmacy benefit plans having different plan sponsors or clients and/or for different prescription drugs. The member's copayment may be a flat copayment (in one example, $10), coinsurance (in one example, 10%), and/or a deductible (for example, responsibility for the first $500 of annual prescription drug expense, etc.) for certain prescription drugs, certain types and/or classes of prescription drugs, and/or all prescription drugs. The copayment may be stored in a storage device 110 or determined by the benefit manager device 102.

In some instances, the member may not pay the copayment or may only pay a portion of the copayment for the prescription drug. For example, if a usual and customary cost for a generic version of a prescription drug is $4, and the member's flat copayment is $20 for the prescription drug, the member may only need to pay $4 to receive the prescription drug. In another example involving a worker's compensation claim, no copayment may be due by the member for the prescription drug.

In addition, copayments may also vary based on different delivery channels for the prescription drug. For example, the copayment for receiving the prescription drug from a mail order pharmacy location may be less than the copayment for receiving the prescription drug from a retail pharmacy location.

In conjunction with receiving a copayment (if any) from the member and dispensing the prescription drug to the member, the pharmacy submits a claim to the PBM for the prescription drug. After receiving the claim, the PBM (such as by using the benefit manager device 102) may perform certain adjudication operations including verifying eligibility for the member, identifying/reviewing an applicable formulary for the member to determine any appropriate copayment, coinsurance, and deductible for the prescription drug, and performing a drug utilization review (DUR) for the member. Further, the PBM may provide a response to the pharmacy (for example, the pharmacy system 100) following performance of at least some of the aforementioned operations.

As part of the adjudication, a plan sponsor (or the PBM on behalf of the plan sponsor) ultimately reimburses the pharmacy for filling the prescription drug when the prescription drug is successfully adjudicated. The aforementioned adjudication operations generally occur before the copayment is received and the prescription drug is dispensed. However, in some instances, these operations may occur simultaneously, substantially simultaneously, or in a different order. In addition, more or fewer adjudication operations may be performed as at least part of the adjudication process.

The amount of reimbursement paid to the pharmacy by a plan sponsor and/or money paid by the member may be determined at least partially based on types of pharmacy networks in which the pharmacy is included. In some implementations, the amount may also be determined based on other factors. For example, if the member pays the pharmacy for the prescription drug without using the prescription or drug benefit provided by the PBM, the amount of money paid by the member may be higher than when the member uses the prescription or drug benefit. In some implementations, the amount of money received by the pharmacy for dispensing the prescription drug and for the prescription drug itself may be higher than when the member uses the prescription or drug benefit. Some or all of the foregoing operations may be performed by executing instructions stored in the benefit manager device 102 and/or an additional device.

Examples of the network 104 include a Global System for Mobile Communications (GSM) network, a code division multiple access (CDMA) network, 3rd Generation Partnership Project (3GPP), an Internet Protocol (IP) network, a Wireless Application Protocol (WAP) network, or an IEEE 802.11 standards network, as well as various combinations of the above networks. The network 104 may include an optical network. The network 104 may be a local area network or a global communication network, such as the Internet. In some implementations, the network 104 may include a network dedicated to prescription orders: a prescribing network such as the electronic prescribing network operated by Surescripts of Arlington, Virginia.

Moreover, although the system shows a single network 104, multiple networks can be used. The multiple networks may communicate in series and/or parallel with each other to link the devices 102-110.

The pharmacy device 106 may be a device associated with a retail pharmacy location (e.g., an exclusive pharmacy location, a grocery store with a retail pharmacy, or a general sales store with a retail pharmacy) or other type of pharmacy location at which a member attempts to obtain a prescription. The pharmacy may use the pharmacy device 106 to submit the claim to the PBM for adjudication.

Additionally, in some implementations, the pharmacy device 106 may enable information exchange between the pharmacy and the PBM. For example, this may allow the sharing of member information such as drug history that may allow the pharmacy to better service a member (for example, by providing more informed therapy consultation and drug interaction information). In some implementations, the benefit manager device 102 may track prescription drug fulfillment and/or other information for users that are not members, or have not identified themselves as members, at the time (or in conjunction with the time) in which they seek to have a prescription filled at a pharmacy.

The pharmacy device 106 may include a pharmacy fulfillment device 112, an order processing device 114, and a pharmacy management device 116 in communication with each other directly and/or over the network 104. The order processing device 114 may receive information regarding filling prescriptions and may direct an order component to one or more devices of the pharmacy fulfillment device 112 at a pharmacy. The pharmacy fulfillment device 112 may fulfill, dispense, aggregate, and/or pack the order components of the prescription drugs in accordance with one or more prescription orders directed by the order processing device 114.

In general, the order processing device 114 is a device located within or otherwise associated with the pharmacy to enable the pharmacy fulfillment device 112 to fulfill a prescription and dispense prescription drugs. In some implementations, the order processing device 114 may be an external order processing device separate from the pharmacy and in communication with other devices located within the pharmacy.

For example, the external order processing device may communicate with an internal pharmacy order processing device and/or other devices located within the system 100. In some implementations, the external order processing device may have limited functionality (e.g., as operated by a user requesting fulfillment of a prescription drug), while the internal pharmacy order processing device may have greater functionality (e.g., as operated by a pharmacist).

The provider device 109 is a device or system at a provider location that can provide communications to other devices and systems over the network 104. The provider device 109 can store and interact with records at the provider, e.g., patient appointment data, medical diagnosis data, medical plan data, financial data, billing data, diagnosis code data and the like.

The order processing device 114 may track the prescription order as it is fulfilled by the pharmacy fulfillment device 112. The prescription order may include one or more prescription drugs to be filled by the pharmacy. The order processing device 114 may make pharmacy routing decisions and/or order consolidation decisions for the particular prescription order. The pharmacy routing decisions include what device(s) in the pharmacy are responsible for filling or otherwise handling certain portions of the prescription order. The order consolidation decisions include whether portions of one prescription order or multiple prescription orders should be shipped together for a user or a user family. The order processing device 114 may also track and/or schedule literature or paperwork associated with each prescription order or multiple prescription orders that are being shipped together. In some implementations, the order processing device 114 may operate in combination with the pharmacy management device 116.

The order processing device 114 may include circuitry, a processor, a memory to store data and instructions, and communication functionality. The order processing device 114 is dedicated to performing processes, methods, and/or instructions described in this application. Other types of electronic devices may also be used that are specifically configured to implement the processes, methods, and/or instructions described in further detail below.

In some implementations, at least some functionality of the order processing device 114 may be included in the pharmacy management device 116. The order processing device 114 may be in a client-server relationship with the pharmacy management device 116, in a peer-to-peer relationship with the pharmacy management device 116, or in a different type of relationship with the pharmacy management device 116. The order processing device 114 and/or the pharmacy management device 116 may communicate directly (for example, such as by using a local storage) and/or through the network 104 (such as by using a cloud storage configuration, software as a service, etc.) with the storage device 110.

The storage device 110 may include: non-transitory storage (for example, memory, hard disk, CD-ROM, etc.) in communication with the benefit manager device 102 and/or the pharmacy device 106 directly and/or over the network 104. The non-transitory storage may store order data 118, member data 120, claims data 122, drug data 124, prescription data 126, and/or plan sponsor data 128. Further, the system 100 may include additional devices, which may communicate with each other directly or over the network 104.

The order data 118 may be related to a prescription order. The order data may include a type of the prescription drug (for example, drug name and strength) and quantity of the prescription drug. The order data 118 may also include data used for completion of the prescription, such as prescription materials. In general, prescription materials include an electronic copy of information regarding the prescription drug for inclusion with or otherwise in conjunction with the fulfilled prescription. The prescription materials may include electronic information regarding drug interaction warnings, recommended usage, possible side effects, expiration date, date of prescribing, etc. The order data 118 may be used by a high-volume fulfillment center to fulfill a pharmacy order.

In some implementations, the order data 118 includes verification information associated with fulfillment of the prescription in the pharmacy. For example, the order data 118 may include videos and/or images taken of (i) the prescription drug prior to dispensing, during dispensing, and/or after dispensing, (ii) the prescription container (for example, a prescription container and sealing lid, prescription packaging, etc.) used to contain the prescription drug prior to dispensing, during dispensing, and/or after dispensing, (iii) the packaging and/or packaging materials used to ship or otherwise deliver the prescription drug prior to dispensing, during dispensing, and/or after dispensing, and/or (iv) the fulfillment process within the pharmacy. Other types of verification information such as barcode data read from pallets, bins, trays, or carts used to transport prescriptions within the pharmacy may also be stored as order data 118.

The member data 120 includes information regarding the members associated with the PBM. The information stored as member data 120 may include personal information, personal health information, protected health information, etc. Examples of the member data 120 include name, address, telephone number, e-mail address, prescription drug history, etc. The member data 120 may include a plan sponsor identifier that identifies the plan sponsor associated with the member and/or a member identifier that identifies the member to the plan sponsor. The member data 120 may include a member identifier that identifies the plan sponsor associated with the user and/or a user identifier that identifies the user to the plan sponsor. The member data 120 may also include dispensation preferences such as type of label, type of cap, message preferences, language preferences, etc.

The member data 120 may be accessed by various devices in the pharmacy (for example, the high-volume fulfillment center, etc.) to obtain information used for fulfillment and shipping of prescription orders. In some implementations, an external order processing device operated by or on behalf of a member may have access to at least a portion of the member data 120 for review, verification, or other purposes.

In some implementations, the member data 120 may include information for persons who are users of the pharmacy but are not members in the pharmacy benefit plan being provided by the PBM. For example, these users may obtain drugs directly from the pharmacy, through a private label service offered by the pharmacy, the high-volume fulfillment center, or otherwise. In general, the terms “member” and “user” may be used interchangeably.

The claims data 122 includes information regarding pharmacy claims adjudicated by the PBM under a drug benefit program provided by the PBM for one or more plan sponsors. In general, the claims data 122 includes an identification of the client that sponsors the drug benefit program under which the claim is made, and/or the member that purchased the prescription drug giving rise to the claim, the prescription drug that was filled by the pharmacy (e.g., the national drug code number, etc.), the dispensing date, generic indicator, generic product identifier (GPI) number, medication class, the cost of the prescription drug provided under the drug benefit program, the copayment/coinsurance amount, rebate information, and/or member eligibility, etc. Additional information may be included.

In some implementations, other types of claims beyond prescription drug claims may be stored in the claims data 122. For example, medical claims, dental claims, wellness claims, or other types of health-care-related claims for members may be stored as a portion of the claims data 122.

In some implementations, the claims data 122 includes claims that identify the members with whom the claims are associated. Additionally or alternatively, the claims data 122 may include claims that have been de-identified (that is, associated with a unique identifier but not with a particular, identifiable member).

The drug data 124 may include drug name (e.g., technical name and/or common name), other names by which the drug is known, active ingredients, an image of the drug (such as in pill form), etc. The drug data 124 may include information associated with a single medication or multiple medications.

The prescription data 126 may include information regarding prescriptions that may be issued by prescribers on behalf of users, who may be members of the pharmacy benefit plan—for example, to be filled by a pharmacy. Examples of the prescription data 126 include user names, medication or treatment (such as lab tests), dosing information, etc. The prescriptions may include electronic prescriptions or paper prescriptions that have been scanned. In some implementations, the dosing information reflects a frequency of use (e.g., once a day, twice a day, before each meal, etc.) and a duration of use (e.g., a few days, a week, a few weeks, a month, etc.).

In some implementations, the order data 118 may be linked to associated member data 120, claims data 122, drug data 124, and/or prescription data 126.

The plan sponsor data 128 includes information regarding the plan sponsors of the PBM. Examples of the plan sponsor data 128 include company name, company address, contact name, contact telephone number, contact e-mail address, etc.

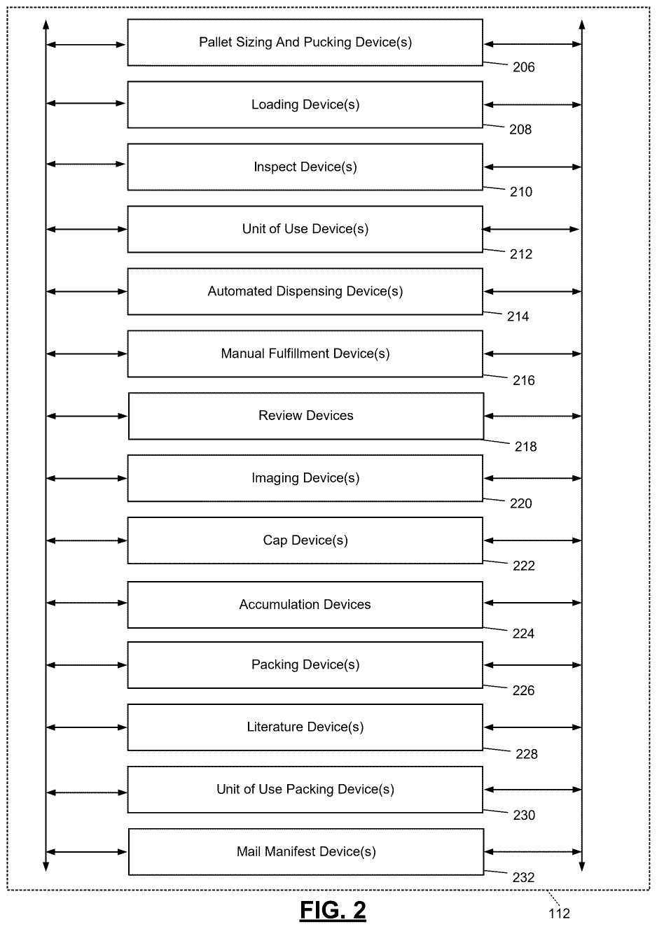

FIG. 2 illustrates the pharmacy fulfillment device 112 according to an example implementation. The pharmacy fulfillment device 112 may be used to process and fulfill prescriptions and prescription orders. After fulfillment, the fulfilled prescriptions are packed for shipping.

The pharmacy fulfillment device 112 may include devices in communication with the benefit manager device 102, the order processing device 114, and/or the storage device 110, directly or over the network 104. Specifically, the pharmacy fulfillment device 112 may include pallet sizing and pucking device(s) 206, loading device(s) 208, inspect device(s) 210, unit of use device(s) 212, automated dispensing device(s) 214, manual fulfillment device(s) 216, review devices 218, imaging device(s) 220, cap device(s) 222, accumulation devices 224, packing device(s) 226, literature device(s) 228, unit of use packing device(s) 230, and mail manifest device(s) 232. Further, the pharmacy fulfillment device 112 may include additional devices, which may communicate with each other directly or over the network 104.

In some implementations, operations performed by one of these devices 206-232 may be performed sequentially, or in parallel with the operations of another device as may be coordinated by the order processing device 114. In some implementations, the order processing device 114 tracks a prescription with the pharmacy based on operations performed by one or more of the devices 206-232.

In some implementations, the pharmacy fulfillment device 112 may transport prescription drug containers, for example, among the devices 206-232 in the high-volume fulfillment center, by use of pallets. The pallet sizing and pucking device 206 may configure pucks in a pallet. A pallet may be a transport structure for a number of prescription containers, and may include a number of cavities. A puck may be placed in one or more than one of the cavities in a pallet by the pallet sizing and pucking device 206. The puck may include a receptacle sized and shaped to receive a prescription container. Such containers may be supported by the pucks during carriage in the pallet. Different pucks may have differently sized and shaped receptacles to accommodate containers of differing sizes, as may be appropriate for different prescriptions.

The arrangement of pucks in a pallet may be determined by the order processing device 114 based on prescriptions that the order processing device 114 decides to launch. The arrangement logic may be implemented directly in the pallet sizing and pucking device 206. Once a prescription is set to be launched, a puck suitable for the appropriate size of container for that prescription may be positioned in a pallet by a robotic arm or pickers. The pallet sizing and pucking device 206 may launch a pallet once pucks have been configured in the pallet.

The loading device 208 may load prescription containers into the pucks on a pallet by a robotic arm, a pick and place mechanism (also referred to as pickers), etc. In various implementations, the loading device 208 has robotic arms or pickers to grasp a prescription container and move it to and from a pallet or a puck. The loading device 208 may also print a label that is appropriate for a container that is to be loaded onto the pallet, and apply the label to the container. The pallet may be located on a conveyor assembly during these operations (e.g., at the high-volume fulfillment center, etc.).

The inspect device 210 may verify that containers in a pallet are correctly labeled and in the correct spot on the pallet. The inspect device 210 may scan the label on one or more containers on the pallet. Labels of containers may be scanned or imaged in full or in part by the inspect device 210. Such imaging may occur after the container has been lifted out of its puck by a robotic arm, picker, etc., or may be otherwise scanned or imaged while retained in the puck. In some implementations, images and/or video captured by the inspect device 210 may be stored in the storage device 110 as order data 118.

The unit of use device 212 may temporarily store, monitor, label, and/or dispense unit of use products. In general, unit of use products are prescription drug products that may be delivered to a user or member without being repackaged at the pharmacy. These products may include pills in a container, pills in a blister pack, inhalers, etc. Prescription drug products dispensed by the unit of use device 212 may be packaged individually or collectively for shipping, or may be shipped in combination with other prescription drugs dispensed by other devices in the high-volume fulfillment center.

At least some of the operations of the devices 206-232 may be directed by the order processing device 114. For example, the manual fulfillment device 216, the review device 218, the automated dispensing device 214, and/or the packing device 226, etc. may receive instructions provided by the order processing device 114.

The automated dispensing device 214 may include one or more devices that dispense prescription drugs or pharmaceuticals into prescription containers in accordance with one or multiple prescription orders. In general, the automated dispensing device 214 may include mechanical and electronic components with, in some implementations, software and/or logic to facilitate pharmaceutical dispensing that would otherwise be performed in a manual fashion by a pharmacist and/or pharmacist technician. For example, the automated dispensing device 214 may include high-volume fillers that fill a number of prescription drug types at a rapid rate and blister pack machines that dispense and pack drugs into a blister pack. Prescription drugs dispensed by the automated dispensing devices 214 may be packaged individually or collectively for shipping, or may be shipped in combination with other prescription drugs dispensed by other devices in the high-volume fulfillment center.

The manual fulfillment device 216 controls how prescriptions are manually fulfilled. For example, the manual fulfillment device 216 may receive or obtain a container and enable fulfillment of the container by a pharmacist or pharmacy technician. In some implementations, the manual fulfillment device 216 provides the filled container to another device in the pharmacy fulfillment devices 112 to be joined with other containers in a prescription order for a user or member.

In general, manual fulfillment may include operations at least partially performed by a pharmacist or a pharmacy technician. For example, a person may retrieve a supply of the prescribed drug, may make an observation, may count out a prescribed quantity of drugs and place them into a prescription container, etc. Some portions of the manual fulfillment process may be automated by use of a machine. For example, counting of capsules, tablets, or pills may be at least partially automated (such as through use of a pill counter). Prescription drugs dispensed by the manual fulfillment device 216 may be packaged individually or collectively for shipping, or may be shipped in combination with other prescription drugs dispensed by other devices in the high-volume fulfillment center.

The review device 218 may process prescription containers to be reviewed by a pharmacist for proper pill count, exception handling, prescription verification, etc. Fulfilled prescriptions may be manually reviewed and/or verified by a pharmacist, as may be required by state or local law. A pharmacist or other licensed pharmacy person who may dispense certain drugs in compliance with local and/or other laws may operate the review device 218 and visually inspect a prescription container that has been filled with a prescription drug. The pharmacist may review, verify, and/or evaluate drug quantity, drug strength, and/or drug interaction concerns, or otherwise perform pharmacist services. The pharmacist may also handle containers which have been flagged as an exception, such as containers with unreadable labels, containers for which the associated prescription order has been canceled, containers with defects, etc. In an example, the manual review can be performed at a manual review station.

The imaging device 220 may image containers once they have been filled with pharmaceuticals. The imaging device 220 may measure a fill height of the pharmaceuticals in the container based on the obtained image to determine if the container is filled to the correct height given the type of pharmaceutical and the number of pills in the prescription. Images of the pills in the container may also be obtained to detect the size of the pills themselves and markings thereon. The images may be transmitted to the order processing device 114 and/or stored in the storage device 110 as part of the order data 118.

The cap device 222 may be used to cap or otherwise seal a prescription container. In some implementations, the cap device 222 may secure a prescription container with a type of cap in accordance with a user preference (e.g., a preference regarding child resistance, etc.), a plan sponsor preference, a prescriber preference, etc. The cap device 222 may also etch a message into the cap, although this process may be performed by a subsequent device in the high-volume fulfillment center.

The accumulation device 224 accumulates various containers of prescription drugs in a prescription order. The accumulation device 224 may accumulate prescription containers from various devices or areas of the pharmacy. For example, the accumulation device 224 may accumulate prescription containers from the unit of use device 212, the automated dispensing device 214, the manual fulfillment device 216, and the review device 218. The accumulation device 224 may be used to group the prescription containers prior to shipment to the member.

The literature device 228 prints, or otherwise generates, literature to include with each prescription drug order. The literature may be printed on multiple sheets of substrates, such as paper, coated paper, printable polymers, or combinations of the above substrates. The literature printed by the literature device 228 may include information required to accompany the prescription drugs included in a prescription order, other information related to prescription drugs in the order, financial information associated with the order (for example, an invoice or an account statement), etc.

In some implementations, the literature device 228 folds or otherwise prepares the literature for inclusion with a prescription drug order (e.g., in a shipping container). In other implementations, the literature device 228 prints the literature and is separate from another device that prepares the printed literature for inclusion with a prescription order.

The packing device 226 packages the prescription order in preparation for shipping the order. The packing device 226 may box, bag, or otherwise package the fulfilled prescription order for delivery. The packing device 226 may further place inserts (e.g., literature or other papers, etc.) into the packaging received from the literature device 228. For example, bulk prescription orders may be shipped in a box, while other prescription orders may be shipped in a bag, which may be a wrap seal bag.

The packing device 226 may label the box or bag with an address and a recipient's name. The label may be printed and affixed to the bag or box, be printed directly onto the bag or box, or otherwise associated with the bag or box. The packing device 226 may sort the box or bag for mailing in an efficient manner (e.g., sort by delivery address, etc.). The packing device 226 may include ice or temperature sensitive elements for prescriptions that are to be kept within a temperature range during shipping (for example, this may be necessary in order to retain efficacy). The ultimate package may then be shipped through postal mail, through a mail order delivery service that ships via ground and/or air (e.g., UPS, FEDEX, or DHL, etc.), through a delivery service, through a locker box at a shipping site (e.g., AMAZON locker or a PO Box, etc.), or otherwise.

The unit of use packing device 230 packages a unit of use prescription order in preparation for shipping the order. The unit of use packing device 230 may include manual scanning of containers to be bagged for shipping to verify each container in the order. In an example implementation, the manual scanning may be performed at a manual scanning station. The pharmacy fulfillment device 112 may also include a mail manifest device 232 to print mailing labels used by the packing device 226 and may print shipping manifests and packing lists.

While the pharmacy fulfillment device 112 in FIG. 2 is shown to include single devices 206-232, multiple devices may be used. When multiple devices are present, the multiple devices may be of the same device type or models, or may be a different device type or model. The types of devices 206-232 shown in FIG. 2 are example devices. In other configurations of the system 100, lesser, additional, or different types of devices may be included.

Moreover, multiple devices may share processing and/or memory resources. The devices 206-232 may be located in the same area or in different locations. For example, the devices 206-232 may be located in a building or set of adjoining buildings. The devices 206-232 may be interconnected (such as by conveyors), networked, and/or otherwise in contact with one another or integrated with one another (e.g., at the high-volume fulfillment center, etc.). In addition, the functionality of a device may be split among a number of discrete devices and/or combined with other devices.

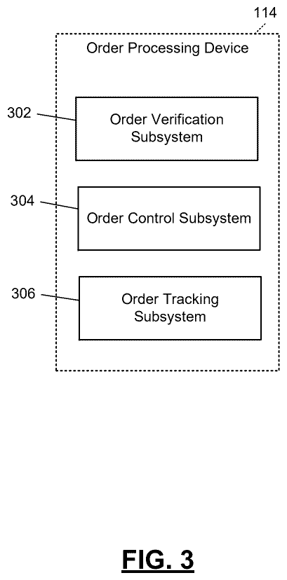

FIG. 3 illustrates the order processing device 114 according to an example implementation. The order processing device 114 may be used by one or more operators to generate prescription orders, make routing decisions, make prescription order consolidation decisions, track literature with the system 100, and/or view order status and other order related information. For example, the prescription order may be comprised of order components.

The order processing device 114 may receive instructions to fulfill an order without operator intervention. An order component may include a prescription drug fulfilled by use of a container through the system 100. The order processing device 114 may include an order verification subsystem 302, an order control subsystem 304, and/or an order tracking subsystem 306. Other subsystems may also be included in the order processing device 114.

The order verification subsystem 302 may communicate with the benefit manager device 102 to verify the eligibility of the member and review the formulary to determine appropriate copayment, coinsurance, and deductible for the prescription drug and/or perform a DUR (drug utilization review). Other communications between the order verification subsystem 302 and the benefit manager device 102 may be performed for a variety of purposes.

The order control subsystem 304 controls various movements of the containers and/or pallets along with various filling functions during their progression through the system 100. In some implementations, the order control subsystem 304 may identify the prescribed drug in one or more than one prescription orders as capable of being fulfilled by the automated dispensing device 214. The order control subsystem 304 may determine which prescriptions are to be launched and may determine that a pallet of automated-fill containers is to be launched.

The order control subsystem 304 may determine that an automated-fill prescription of a specific pharmaceutical is to be launched and may examine a queue of orders awaiting fulfillment for other prescription orders, which will be filled with the same pharmaceutical. The order control subsystem 304 may then launch orders with similar automated-fill pharmaceutical needs together in a pallet to the automated dispensing device 214. As the devices 206-232 may be interconnected by a system of conveyors or other container movement systems, the order control subsystem 304 may control various conveyors: for example, to deliver the pallet from the loading device 208 to the manual fulfillment device 216 from the literature device 228, paperwork as needed to fill the prescription.

The order tracking subsystem 306 may track a prescription order during its progress toward fulfillment. The order tracking subsystem 306 may track, record, and/or update order history, order status, etc. The order tracking subsystem 306 may store data locally (for example, in a memory) or as a portion of the order data 118 stored in the storage device 110.

### Medical Appointment Swap Management System

In some example embodiments described herein, a patient may elect to swap an appointment for an appointment slot of another patient. For example, patients may opt in to an appointment swap availability system, and select criteria for permitting swaps. Example criteria may include, but are not limited to, a date or range of dates, a day or range of days, a time or range of times, one or more providers, one or more practices, one or more provider specialties, a provider gender, fill of prescription medications by the pharmacy described herein, adherence to drug treatment using prescriptions filled by the pharmacy, etc.

An example system may match other patients' appointments (e.g., who have also opted in) against the specified criteria, and propose a swap (e.g., without sharing protected health information (PHI) or personal identifiable information (PII)). In some examples, both patients must agree to the swap before the swap can occur. In some cases, the provider system must also agree to the swap, and may block a swap based on a medical basis. For example, certain types of appointments may be blocked from swapping, such as a first point surgery visit. Swapping may occur only if a provider matches patient preferences.

Some systems may implement a best match appointment scheduling feature. For example, artificial intelligence (AI) may be used to select patients to opt into the swap system or to opt out of the swap system. In some examples, generative AI, machine learning models, etc., may be configured to select possible swap time and date slots based on historical appointments, with a current provider and other providers.

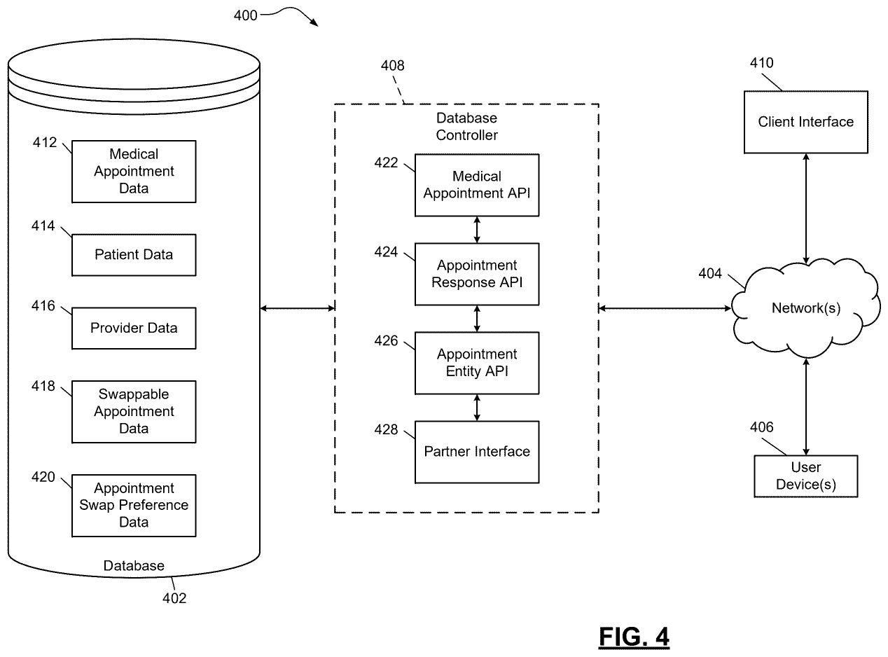

FIG. 4 is a functional block diagram of an example system 400 for medical appointment swap management, which includes a database 402. While the system 400 is generally described as being deployed in a computer network system, the database 402 and/or components of the system 400 may otherwise be deployed (for example, as a standalone computer setup). The system 400 may include a desktop computer, a laptop computer, a tablet, a smartphone, etc., which when loaded with the instructions related to methods described herein are dedicated machines for the presently described embodiments of the invention and its equivalents.

As shown in FIG. 4, the database 402 stores medical appointment data 412, patient data 414, provider data 416, swappable appointment data 418, and appointment swap preference data 420. In various implementations, the database 402 may store other types of data as well. The medical appointment data 412, patient data 414, provider data 416, swappable appointment data 418, and appointment swap preference data 420 may be located in different physical memories within the database 402, such as different random access memory (RAM), read-only memory (ROM), a non-volatile hard disk or flash memory, etc. For example, some data may be stored on servers of a third party vendor.

In some implementations, the medical appointment data 412, patient data 414, provider data 416, swappable appointment data 418, and appointment swap preference data 420 may be located in the same memory (such as in different address ranges of the same memory). In various implementations, the medical appointment data 412, patient data 414, provider data 416, swappable appointment data 418, and appointment swap preference data 420 may each be stored as structured data in any suitable type of data store.

The medical appointment data 412 may include any suitable data regarding medical appointment slots, such as times and dates associated with medical appointments, providers and locations associated with medical appointments, available or booked status values for medical appointments, etc.

The patient data 414 may include any suitable data regarding patient information, such as demographic information, billing and payment history, claims data, insurance information, past medical data, present medical data, past prescription data, present prescription data, and diagnoses, etc. The patient data 414 may be stored in an electronic or other machine-readable format with multiple fields (e.g., data types), that can be selectably displayed on a screen via a graphical user interface. Patient data fields can include diagnoses, cardiological, radiology, pre-operative, operative, post-operative, reports, discharge, and follow up. Each of these data fields can be displayed as a table if there are multiple entries. As described herein, the data in the patient data fields can be used to prevent a patient from agreeing to swapping an appointment to a later time based on rules associated with the data in the patient record. For example, certain cardiology data or post-operative care can trigger a flag that prevents a patient from swapping a medical appointment. In a further example, the flag in the patient record or the patient appointment record can either allow a medical visit swap or block a proposed appointment swap. Rules that prevent swaps by setting the flag can be dynamic and set by a provider. A large language model may be employed to determine the types of patient records that are eligible for appointment swapping.

The provider data 416 may include any suitable data associated with medical care providers, such as doctor names, locations, specialties and types of medical practices, etc. The swappable appointment data 418 may include any suitable data associated with swap statuses of appointments, such as whether booked appointments have a status indicating that the patient currently holding the appointment is willing to swap with another patient desiring the same appointment slot, etc.

The appointment swap preference data 420 may include preferences of patients, such as whether they are willing to swap a booked appointment slot, whether they accept or decline a request to swap for a booked appointment slot from another patient, etc. For example, patients may request to swap for booked appointment slots by accessing the system controller 408 via the user device 406. The user device 406 may include any suitable user device for displaying text and receiving input from a user, including a desktop computer, a laptop computer, a tablet, a smartphone, etc. In various implementations, the user device 406 may access the database 402 or the system controller 408 directly, or may access the database 402 or the system controller 408 through one or more networks 404. Example networks may include a wireless network, a local area network (LAN), the Internet, a cellular network, etc.

The system controller 408 may include one or more modules for automated entity field correction. For example, FIG. 4 illustrates a medical appointment application programing interface (API) 422, an appointment response API 424, an appointment entity API 426, and a partner interface 428. The medical appointment API 408 may be configured to receive requests for desired medical appointment slots, such as from a client interface 410. The APIs can be produced by the models, large language model or generative artificial intelligence as described herein.

The appointment response API 424 may be configured to supply responses (e.g., to the client interface 410), such as providing a notification of availability regarding a desired appointment slot, updating a status of an appointment slot to booked, etc. The appointment entity API 426 may be configured to modify stored values in an appointment slot table, such as adding details or preferences to a swappable appointment table, or receiving a request for a swappable appointment. The partner interface 428 may be configured to book slots on a waitlist, provide an acceptance or declined status in response to a swap request, etc. Some of the modules may be part of, or configured to communicate with, a third party vendor.

In some examples, the system may be configured to provide an ability for physicians to refer patients based on diagnosis/issue/concern to a specialist. The system may automate a referrals process based on electronic health records (HER), geo-proximity to a patient, convenience, defaulting to network preferred providers (e.g., INN specialist), etc. An algorithm may be built to identify what specialist to refer a patient to, which may include analyzing historical records to find preferred appointment times. This may be implemented as a pharmacy benefit manager (PBM) solution, such as a default preference for members.

In some implementations, the system may be configured to provide an ability to request appointment times based on patient historical appointments, in a priority order. For example, the system may analyze patient appointments so when the patient goes to create a new appointment, asks to be on waitlist or to swap appointments, AI may serve up and auto-fill appointment times based on, e.g., appointment history, type of appointment, day, time of appointment, etc. The system may allow patients to accept/decline, generate an alert if a preference still works for the patient, navigate a user to update preferences, allow a patient to pick appointments or preference manually, etc. In some cases, the system may generate a reminder for a patient to update their preferences periodically. The system may be configured to analyze patients over different historical timeframes, such as one year, etc., which may include analysis of specified types of appointments, specified physicians, etc.

In some examples, the system may be configured to provide an ability to set up recurring appointments based on a condition, disease state, comorbidity diagnosis, etc. For example, the system may analyze patient historical times to apply day/time criteria per appointment. The system may help guide a patient through ha care plan journey, including predicting preferences for patients based on historical appointment history. The system may allow a user to select preferences for the system to learn from. A caregiver which provides support on behalf of patients may be able to input their preferences on behalf of the patient. The system may allow for the ability to add a transportation request, such as a ride-sharing service, a shuttle, etc. The system may predict preferences for patients with scheduling flexibility. Physicians may accommodate recurring appointments, which may require feeding availability of slots from a provider office.

In some implementations, the system may be configured to provide an ability to provide follow up appointments based on historical data, to ensure a patient is guided to the care they need. For example, after an appointment, a care team may provide a recommendation on a follow up appointment based on a patient type, a time need, a virtual versus in-person preference, etc. An urgent care or emergency room (ER) follow up may be scheduled, with a recommended care path forward with a type of specialist and then PCP. The system may specify an order for a type of physician, with appointments to help ensure the patient gets follow up care that they need after they are discharged. The system may help with ER follow ups, and redirect patients to help find a physician they need to lower ER remissions.

In some examples, the system may be configured to provide an ability to prioritize appointments for physicians based on an appointment type, where patient appointments are requested simultaneously. For example, the system may analyze appointments for a physician office, where patients want the same doctor, location, time, etc., and recommend a selected patient as having a highest priority for that time (e.g., based on a type of care needed). The system may be configured to objectively triage for acuity and urgency of patient needs, as compared to subjective decision making.

For example, the system may suggest a next available appointment to a less urgent patient, to give way to a more urgent patient to confirm the appointment time, let a patient know the waiting time is longer than expected, send an appointment to confirm (such as later in the same day, or a same time the next day), etc. The prioritization may be applied to any suitable appointment types, such as dental, ER visits, PCP, urgent care, etc. The system may determine which days and times are more frequently booked, and may adjust for patient convenience. For example, the system may learn how physicians decide patient severity and prioritize who to see sooner.

In some implementations, the system may provide the ability to pull in existing appointments from a stored user calendar, and change preferences based on a patient's current availability. For example, the system may synchronize to a calendar with preferences being offered to the patient, giving the patient only options for open slots on their calendar. The system may also synchronize with booking availability of physicians, to ensure both parties are available. This may include third party integration, which can support households, families, caregivers, shared calendars, etc.

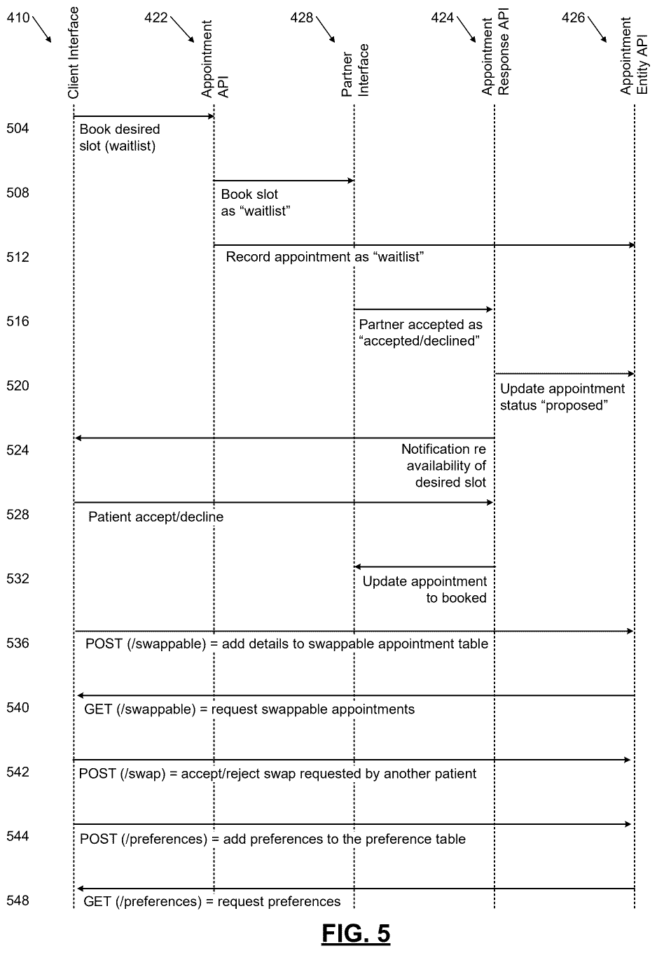

FIG. 5 is a message sequence chart illustrating example interactions between the client interface 410, the appointment API 422, the partner interface 428, the appointment response API 424, and the appointment entity API 426. At line 504, the client interface 402 is configured to transmit a request to book a desired slot to the appointment API 422, which may be include a request to join a waitlist for a desired appointment slot that is currently booked.

At line 508, the appointment API 422 books a slot as “waitlist” with the partner interface 428. At line 512, the appointment API 422 is configured to record the appointment as having a “waitlist” status with the appointment entity API 426. The patient may also book an alternative appointment slot while on the waitlist for a preferred appointment slot.

At line 516, the partner interface 428 supplies a partner acceptance status as “accepted” or “declined” to the appointment response API 424. At line 520, the appointment response API 424 updates the appointment status to “proposed” with the appointment entity API 426.

At line 524, the appointment response API 424 transmits a notification regarding availability of a desired slot to the client interface 410. For example, the notification may be generated in response to a first patient cancelling an appointment slot where a second patient has submitted a waitlist or swap request for that appointment slot.

At line 528, the client interface 410 transmits a patient accept or decline status to the appointment response API 424. For example, a patient may be notified of an available appointment slot they had a waitlist request for, and the patient can decide if they would not like to book the available appointment. At line 532, the appointment response API updates the appointment to booked with the partner interface 428.

At line 536, the client interface 410 executes a POST (/swappable) instruction to the appointment entity API 426, which may add details to a swappable appointment table. For example, this may include an update swappable appointments request via a user interface. At line 540, the client interface executes a GET (/swappable) instruction to the appointment entity API 426, which may include a request for swappable appointments from the table. For example, this may include a fetch swappable appointments request via a user interface. At line 542, the client interface 410 may execute a POST (/swap) instruction to the appointment entity API, to accept or reject a swap requested by another patient.

At line 544, the client interface 410 executes a POST (/preferences) instruction to the appointment entity API 426, which may include adding preferences to a swap preference table. For example, this may include an update preferences request via a user interface. At line 528, the client interface 410 executes a GET (/preferences) instruction to the appointment entity API 426, which may include a request for swap preferences from the table. For example, this may include a fetch preferences request via a user interface.

In the examples described above, any of the lines of the message sequence chart may include an acknowledgment request, a success confirmation message transmission, etc., which is sent back to the requesting module after execution by a target module.

### Medical Appointment Swap Management Processes

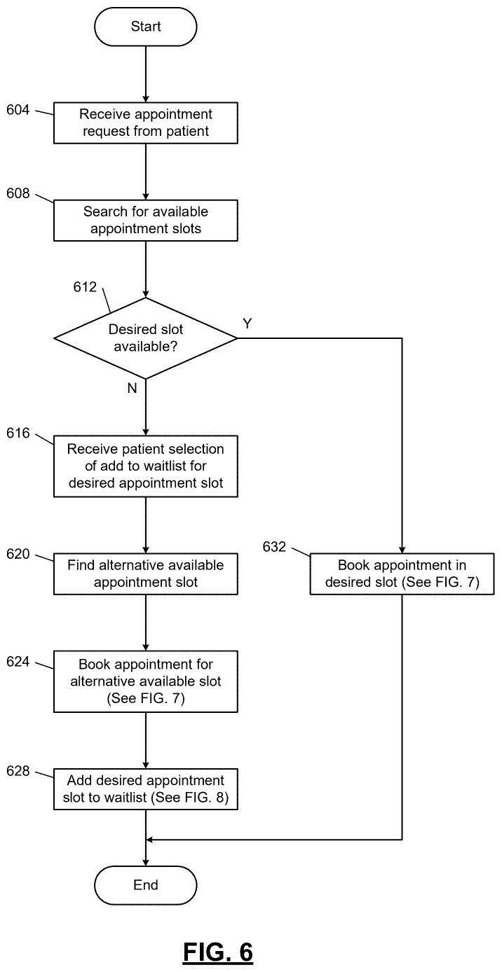

FIG. 6 is a flowchart depicting an example process for requesting a medical appointment using an interface with a medical appointment scheduling system. In various implementations, the process of FIG. 6 may be performed by one or more of the modules of the system controller 408 of FIG. 4.

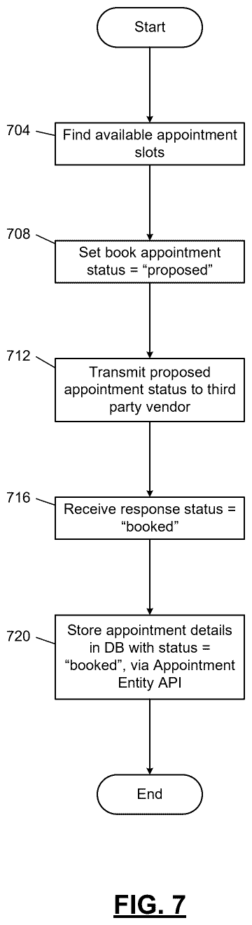

At 604, the process begins by receiving an appointment request from a patient. At 608, control searches for available appointment slots. Control then determines whether any desired slots are available at 612. If so, control proceeds to 632 to book an appointment in the desired slot. Further example details regarding booking appointments and available desired slots are described below with reference to FIG. 7.

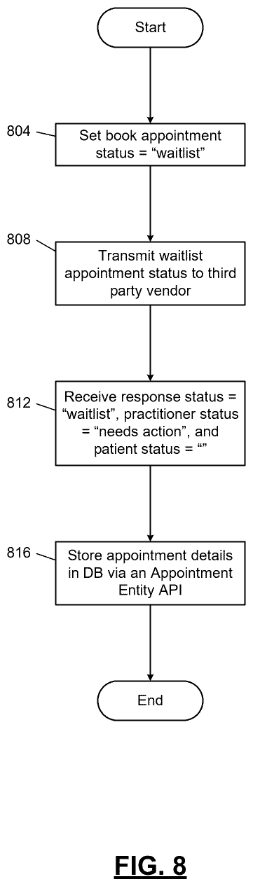

If the desired slot is not available at 612, control proceeds to 616 to receive a patient selection of “add to wait list” for the desired appointment slot. Control then finds an alternative available appointment slot at 620, and books an appointment for the alternative available slot at 624. Control then adds the desired appointment slot to a wait list at 628. Further details regarding adding a desired appointment slot to a wait list are described below with reference to FIG. 8.

FIG. 7 is a flowchart depicting an example process for booking a desired available appointment slot. In various implementations, the process of FIG. 7 may be performed by one or more of the modules of the system controller 408 of FIG. 4.

At 704, the process begins by finding available appointment slots. Control then sets a book appointment status value equal to “proposed” at 708. At 712, control transmits the proposed appointment status to a third party vendor. At 716, control receives a response status value equal to “booked.” Control then stores appointment details in a database at 720, with the status equal to “booked,” via an appointment entity API.

FIG. 8 is a flowchart depicting an example process for adding a request for an unavailable appointment slot to a waitlist. In various implementations, the process of FIG. 8 may be performed by one or more of the modules of the system controller 408 of FIG. 4.

At 804, the process begins by setting a book appointment status equal to “wait list.” Control then transmits the waitlist appointments to a third party vendor at 808. At 812, control receives a response status equal to “wait list,” a new practitioner status equal to “needs action,” and a patient status of “blank” or “null”. Control then stores appointment details in a database at 816 via an appointment entity API.

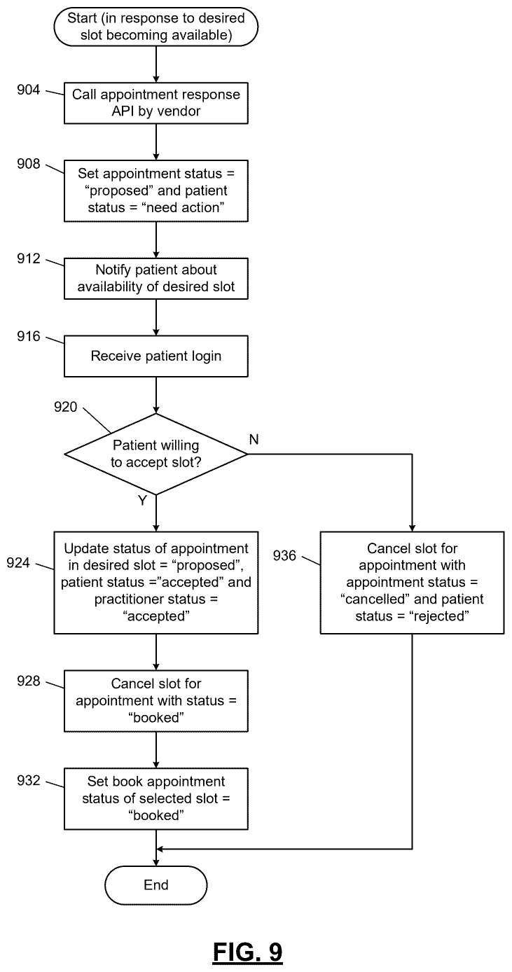

FIG. 9 is a flowchart depicting an example process for revolving waitlisted appointment requests in response to a desired appointment slot becoming available. In various implementations, the process of FIG. 9 may be performed by one or more of the modules of the system controller 408 of FIG. 4.

At 904, control calls an appointment response API via a vendor. This process may occur in response to a desired slot becoming available, such as when a different patient cancels or releases a previously booked appointment slot. At 908, control sets an appointment status as equal to “proposed,” and a patient status equal to “needs action.”

Control then notifies the patient about the availability of the desired slot at 912. The patient notification may be any suitable notification format, such as an e-mail, message, phone call, etc., and may be based on previously specified user preferences.

At 916, control receives a patient login. The patient login information may include a username associated with the patient, a password, two-factor authentication, etc. At 920, control determines whether the patient is willing to accept the slot. For example, the available slot may be presented to the patient on a user interface of a patient device, with the option for the patient to select the newly available slot, or decline the newly available slot.

If the patient is not willing to accept the slot at 920, control proceeds to 936 to cancel the appointment slot with an appointment status equal to “cancelled,” and the patient status equal to “rejected.” If the patient is willing to accept the slot at 920, control proceeds to 924 to update the status of the appointment in the desired slot to “proposed,” update a patient status to “accepted,” and update a practitioner status to “accepted.” Control and cancels the slot for the appointment having the status equal to “booked,” at 928, and sets the book appointment status of the newly selected appointment slot equal to “booked” at 932.

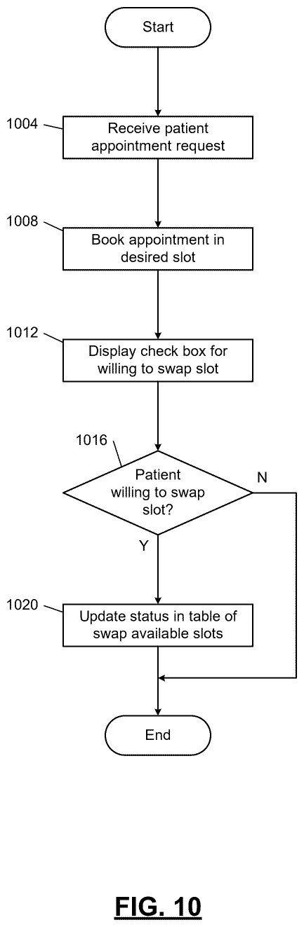

FIG. 10 is a flowchart depicting an example process for checking a status of swap willingness for a patient with a booked appointment. In various implementations, the process of FIG. 10 may be performed by one or more of the modules of the system controller 408 of FIG. 4.

At 1004, the process begins by receiving a patient appointment request. Control then proceeds to 1008 to book an appointment in the desired slot. At 1012, control displays a check box for the user to indicate whether they're willing to swap the appointment slot (e.g., such as in the case where another patient requests to swap for the slot due to a more urgent medical need). In other examples, input selection methods other than check boxes may be used.

At 1016, control determines whether the patient is willing to swap the slot, such as whether the patient checked the box indicating willingness to offer the appointment slot for swapping for other interested patients. If the patient indicated willingness to swap a slot at 1016, control proceeds to 1020 to update a status in a table of appointment slots having swap availability.

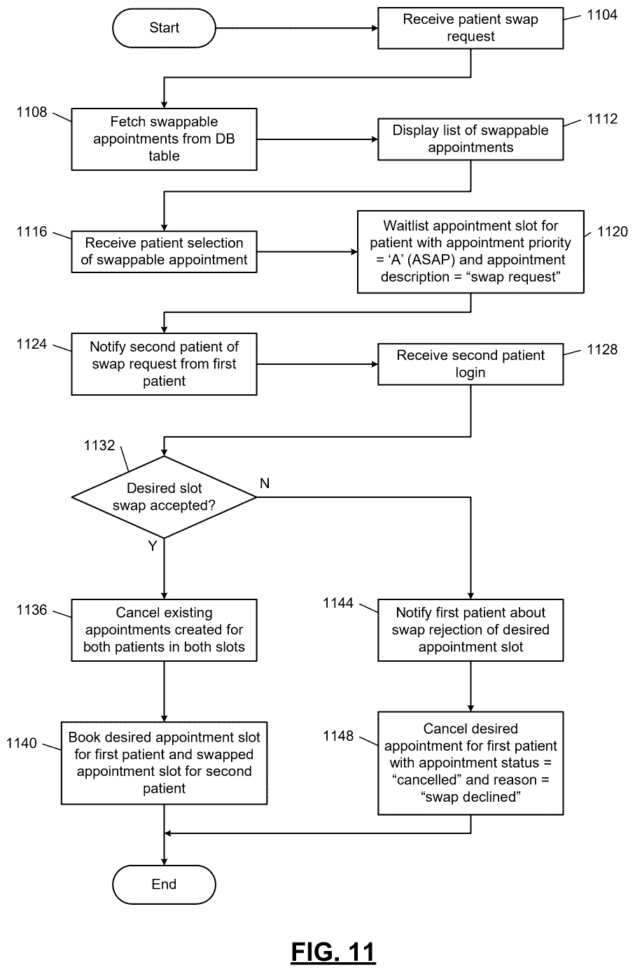

FIG. 11 is a flowchart depicting an example process for swapping a booked appointment slot in response to a swap request from a patient. In various implementations, the process of FIG. 11 may be performed by one or more of the modules of the system controller 408 of FIG. 4.

At 1104, control begins the process by receiving a patient swap request. Control then fetches swappable appointments from a database table at 1108. Control displays the list of swappable appointments at 1112, such as on a user interface of a patient device.

At 1116, control receives a patient selection of a swappable appointment. Control then proceeds to 1120 to waitlist an appointment slot for the patient with an appointment priority status set to ‘A’ or some other value indicating an “as soon as possible” (ASAP) priority. The appointment description may be set to “swap request.”

At 1124, control notifies second patient of the swap request received from the first patient. Control then proceeds to 1128 to receive login information from the second patient. At 1132, control determines whether the desired slot swap is accepted. If not, control proceeds to 1144 to notify the first patient about the swap rejection of the desired appointment slot. Control then cancels the desired appointment for the first patient at 1148, with an appointment status set equal to “cancelled,” and a reason set to “swap declined.”

If the desired slot swap request is accepted by the second patient at 1132, control proceeds to 1136 to cancel previously existing appointments for both patients and both slots. Control then books the desired, swapped appointment slot for the first patient, and the previous appointment of the first patient is assigned to the second patient, at 1140.

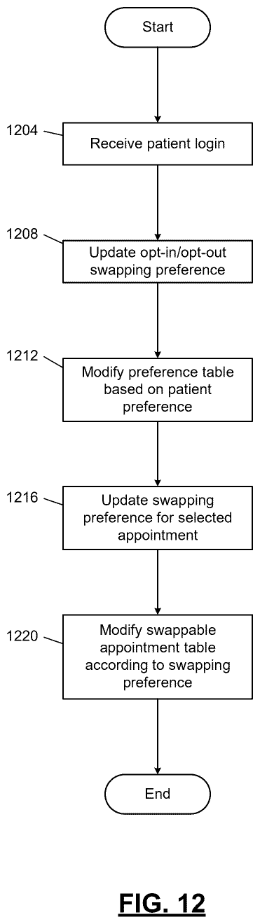

FIG. 12 is a flowchart depicting an example process for modifying a status of opt-in and opt-out swap preferences for a patient. In various implementations, the process of FIG. 12 may be performed by one or more of the modules of the system controller 408 of FIG. 4.

At 1204, control receives a patient login. Control then proceeds to 1208 to update opt-in or opt-out swapping preferences for the patient. At 1212, control modifies the preference table based on patient preferences received from the patient. Control then proceeds to 1216 to update the swapping preference for a selected appointment, and modifies a swappable appointment table at 1220 according to the swapping preference.

### Artificial Intelligence for Swap Management System

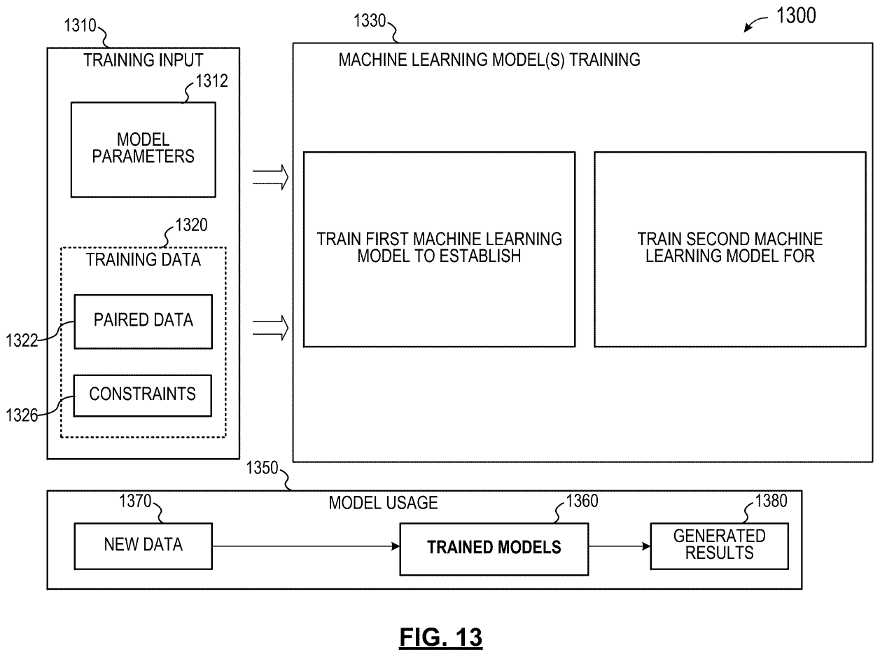

FIG. 13 is a block diagram of an example generative artificial intelligence system 1300 which may be deployed withing the medical appointment swap management of FIG. 1, according to some example embodiments. Training input 1310 includes model parameters 1312 and training data 1320 which may include paired training data sets 1322 (e.g., input-output training pairs) and constraints 1326. Model parameters 1312 stores or provides the parameters or coefficients of corresponding ones of machine learning models. During training, these parameters 1312 are adapted based on the input-output training pairs of the training data sets 1322. After the parameters 1312 are adapted (after training), the parameters are used by trained models 1360 to implement the trained machine learning models on a new set of data 1370.

Training data 1320 includes constraints 1326 which may define the constraints of medical appointment swap features. The paired training data sets 1322 may include sets of input-output pairs. Some components of training input 1310 may be stored separately at a different off-site facility or facilities than other components.

Machine learning model(s) training 1330 trains one or more machine learning techniques based on the sets of input-output pairs of paired training data sets 1322. For example, the model training 1330 may train the machine learning (ML) model parameters 1312 by minimizing a loss function based on one or more ground-truth data.

The ML models can include any one or combination of classifiers or neural networks, such as an artificial neural network, a convolutional neural network, an adversarial network, a generative adversarial network, a deep feed forward network, a radial basis network, a recurrent neural network, a long/short term memory network, a gated recurrent unit, an auto encoder, a variational autoencoder, a denoising autoencoder, a sparse autoencoder, a Markov chain, a Hopfield network, a Boltzmann machine, a restricted Boltzmann machine, a deep belief network, a deep convolutional network, a deconvolutional network, a deep convolutional inverse graphics network, a liquid state machine, an extreme learning machine, an echo state network, a deep residual network, a Kohonen network, a support vector machine, a neural Turing machine, and the like.

After the machine learning models are trained, new data 1370 are received and/or derived. The trained machine learning model may be applied to the new data 1370 to generate results 1380 including a prediction of one or more entities. The model output can be represented in a GUI.

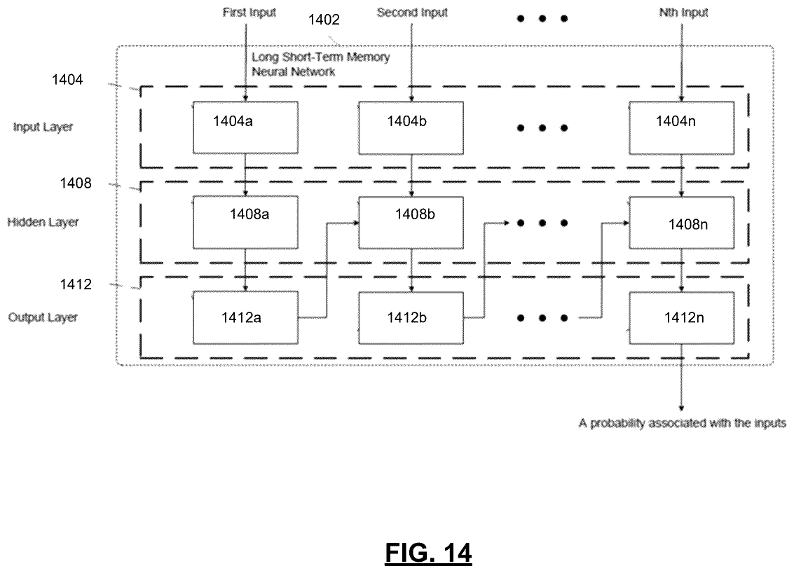

FIG. 14 is a graphical representation of layers or an example long short-term memory (LSTM) machine learning model, which may be used with the medical appointment swap system of FIG. 1 in some example embodiments. In an example, the neural network 1402 can be a LSTM neural network. In an example, the neural network 1402 can be a recurrent neural network (RNN). The example neural network 1402 may be used to implement the machine learning as described herein, and various implementations may use other types of machine learning networks. The neural network 1402 includes an input layer 1404, a hidden layer 1408, and an output layer 1412. The input layer 1404 includes inputs 1404a, 1404b . . . 1404n. The hidden layer 1408 includes neurons 1408a, 1408b . . . 1408n. The output layer 1412 includes outputs 1412a, 1412b . . . 1412n.

Each neuron of the hidden layer 1408 receives an input from the input layer 1404 and outputs a value to the corresponding output in the output layer 1412. For example, the neuron 1408a receives an input from the input 1404a and outputs a value to the output 1412a. Each neuron, other than the neuron 1408a, also receives an output of a previous neuron as an input. For example, the neuron 1408b receives inputs from the input 1404b and the output 1412a. In this way the output of each neuron is fed forward to the next neuron in the hidden layer 1408. The last output 1412n in the output layer 1412 outputs a probability associated with the inputs 1404a-1404n. Although the input layer 1404, the hidden layer 1408, and the output layer 1412 are depicted as each including three elements, each layer may contain any number of elements. Neurons can include one or more adjustable parameters, weights, rules, criteria, or the like.

In various implementations, each layer of the neural network 1402 must include the same number of elements as each of the other layers of the neural network 1402. For example, training features may be processed to create the inputs 1404a-1404n. The inputs can be related to the scheduling of medical appointments, moving medical appointments and medical appointments that cannot be moved.

The neural network 1402 may implement a first model to determine a set of appointments that can be moved, some appointments that cannot be moved, and a set of open appointments. The inputs 1204a-1204n can include one or more of the following: past movement of appointments based on similar patients, similar medical conditions, travel distances, day of week, week of month, availability of provider, standard of care for medical condition, and the like. An input can also include that predicted weather for the appointment time and date. An input can include work hours, school hours of the patient, parent, or legal guardian.

In some examples, a convolutional neural network may be implemented. Similar to neural networks, convolutional neural networks include an input layer, a hidden layer, and an output layer. However, in a convolutional neural network, the output layer includes one fewer output than the number of neurons in the hidden layer and each neuron is connected to each output. Additionally, each input in the input layer is connected to each neuron in the hidden layer. In other words, input 1404a is connected to each of neurons 1408a, 1408b . . . 1408n.

In some examples, a machine learning model is used to process medical data. The use of specially trained machine learning models for processing medical data realizes a number of improvements over traditional methods of medical data processing, including more accurate provider data management, and more accurate processing of medical appointment swaps and associated data. The application further provides methods for training a machine learning model that lead to faster training times and a more accurate model for processing of medical appointment swaps and associated data.

Machine learning models may be used to perform a wide variety of complex tasks, including image recognition, speech recognition, pattern recognition, detection of anomalies, processing of medical data such as medical appointment swaps, etc. A machine learning model may include a biologically inspired algorithm that learns from training data. A machine learning model may be realized through software, hardware, or a combination of software and hardware. The structure of an exemplary machine learning model may have a series of layers, each comprising one or more neurons arranged in one or more neuron arrays.

In some examples, a neuron may comprise a register, a microprocessor, and at least one input. Each neuron produces an output, or activation, based on an activation function that uses the outputs of the previous layer and a set of weights as inputs. Each neuron in a neuron array may be connected to another neuron via a synaptic circuit. A synaptic circuit may include a memory for storing a synaptic weight. An example machine learning model may be a Deep Neural Network having an input layer, an output layer, and a plurality of fully connected hidden layers. Machine learning models are particularly useful in medical data processing because they can effectively extract features in linear and nonlinear relationships.

In some example embodiments, a machine learning model may be implemented by an application-specific integrated circuit (ASIC). ASICs may be specially customized for a specific artificial intelligence application and provide superior computing capabilities and reduced electricity consumption compared to traditional CPUs.

In some examples, training data is generated by receiving continuous data at a computer and using the computer to discretize the continuous data. In some example embodiments, the continuous data may be received remotely over a network. The continuous data may be historical medical data, which the neural network can use to learn patterns to process the data, including for management of medical appointment swaps. Continuous data is data that is measured and can have any number of possible values.

Machine learning models may benefit from being trained with discrete data rather than continuous data. Discrete data can be counted and has a limited number of values. Any type of discretization method may be used to convert continuous data to discrete data, including binning, clustering, and numerical discretization. The machine learning model is then trained using any suitable training techniques to generate a trained neural network which can be used to process medical data.

In some examples, a backpropagation algorithm and a gradient descent algorithm may be used to train the neural network. Gradient descent is an optimization algorithm used to minimize differentiable real-valued multivariate functions. Gradient descent begins by initializing the values of parameters and then applying a gradient descent calculation, which uses mathematical calculations to iteratively adjust the values so they minimize a loss function to optimize the machine learning model.

Backpropagation is the mathematical process of calculating the derivatives and gradient descent is the process of adjusting model parameters using the calculated derivatives to minimize the loss function. Backpropagation is a mathematical calculation for supervised learning of machine learning models using gradient descent. Given a machine learning model and an error function, backpropagation is used to calculate the gradient of the error function with respect to the neural network's weights.

Some example systems may be implemented by one or more processors coupled with one or more non-transitory computer readable media. The methods described herein can be performed by execution of computer-readable instructions stored on a non-transitory computer-readable storage medium (e.g., random-access memory, flash memory, magnetic/optical storage, etc.) by the processor(s). The GUI is hardware or a combination of both hardware and software. The GUI is coupled to the systems described above and is configured to receive user instructions and output model predictions and medical data processing to the user, such as medical appointment swap outputs.

### CONCLUSION

The foregoing description is merely illustrative in nature and is in no way intended to limit the disclosure, its application, or uses. The broad teachings of the disclosure can be implemented in a variety of forms. Therefore, while this disclosure includes particular examples, the true scope of the disclosure should not be so limited since other modifications will become apparent upon a study of the drawings, the specification, and the following claims. In the written description and claims, one or more steps within a method may be executed in a different order (or concurrently) without altering the principles of the present disclosure. Similarly, one or more instructions stored in a non-transitory computer-readable medium may be executed in different order (or concurrently) without altering the principles of the present disclosure. Unless indicated otherwise, numbering or other labeling of instructions or method steps is done for convenient reference, not to indicate a fixed order.

Further, although each of the embodiments is described above as having certain features, any one or more of those features described with respect to any embodiment of the disclosure can be implemented in and/or combined with features of any of the other embodiments, even if that combination is not explicitly described. In other words, the described embodiments are not mutually exclusive, and permutations of one or more embodiments with one another remain within the scope of this disclosure.

Spatial and functional relationships between elements (for example, between modules) are described using various terms, including “connected,” “engaged,” “interfaced,” and “coupled.” Unless explicitly described as being “direct,” when a relationship between first and second elements is described in the above disclosure, that relationship encompasses a direct relationship where no other intervening elements are present between the first and second elements, and also an indirect relationship where one or more intervening elements are present (either spatially or functionally) between the first and second elements.

The phrase “at least one of A, B, and C” should be construed to mean a logical (A OR B OR C), using a non-exclusive logical OR, and should not be construed to mean “at least one of A, at least one of B, and at least one of C.” The term “set” does not necessarily exclude the empty set. The term “non-empty set” may be used to indicate exclusion of the empty set. The term “subset” does not necessarily require a proper subset. In other words, a first subset of a first set may be coextensive with (equal to) the first set.

In the figures, the direction of an arrow, as indicated by the arrowhead, generally demonstrates the flow of information (such as data or instructions) that is of interest to the illustration. For example, when element A and element B exchange a variety of information but information transmitted from element A to element B is relevant to the illustration, the arrow may point from element A to element B. This unidirectional arrow does not imply that no other information is transmitted from element B to element A. Further, for information sent from element A to element B, element B may send requests for, or receipt acknowledgements of, the information to element A.

In this application, including the definitions below, the term “module” or the term “controller” may be replaced with the term “circuit.” The term “module” may refer to, be part of, or include processor hardware (shared, dedicated, or group) that executes code and memory hardware (shared, dedicated, or group) that stores code executed by the processor hardware.

The module may include one or more interface circuits. In some examples, the interface circuit(s) may implement wired or wireless interfaces that connect to a local area network (LAN) or a wireless personal area network (WPAN). Examples of a LAN are Institute of Electrical and Electronics Engineers (IEEE) Standard 802.11-2016 (also known as the WIFI wireless networking standard) and IEEE Standard 802.3-2015 (also known as the ETHERNET wired networking standard). Examples of a WPAN are IEEE Standard 802.15.4 (including the ZIGBEE standard from the ZigBee Alliance) and, from the Bluetooth Special Interest Group (SIG), the BLUETOOTH wireless networking standard (including Core Specification versions 3.0, 4.0, 4.1, 4.2, 5.0, and 5.1 from the Bluetooth SIG).

The module may communicate with other modules using the interface circuit(s). Although the module may be depicted in the present disclosure as logically communicating directly with other modules, in various implementations the module may actually communicate via a communications system. The communications system includes physical and/or virtual networking equipment such as hubs, switches, routers, and gateways. In some implementations, the communications system connects to or traverses a wide area network (WAN) such as the Internet. For example, the communications system may include multiple LANs connected to each other over the Internet or point-to-point leased lines using technologies including Multiprotocol Label Switching (MPLS) and virtual private networks (VPNs).

In various implementations, the functionality of the module may be distributed among multiple modules that are connected via the communications system. For example, multiple modules may implement the same functionality distributed by a load balancing system. In a further example, the functionality of the module may be split between a server (also known as remote, or cloud) module and a client (or, user) module. For example, the client module may include a native or web application executing on a client device and in network communication with the server module.

The term code, as used above, may include software, firmware, and/or microcode, and may refer to programs, routines, functions, classes, data structures, and/or objects. Shared processor hardware encompasses a single microprocessor that executes some or all code from multiple modules. Group processor hardware encompasses a microprocessor that, in combination with additional microprocessors, executes some or all code from one or more modules. References to multiple microprocessors encompass multiple microprocessors on discrete dies, multiple microprocessors on a single die, multiple cores of a single microprocessor, multiple threads of a single microprocessor, or a combination of the above.

Shared memory hardware encompasses a single memory device that stores some or all code from multiple modules. Group memory hardware encompasses a memory device that, in combination with other memory devices, stores some or all code from one or more modules.

The term memory hardware is a subset of the term computer-readable medium. The term computer-readable medium, as used herein, does not encompass transitory electrical or electromagnetic signals propagating through a medium (such as on a carrier wave); the term computer-readable medium is therefore considered tangible and non-transitory. Non-limiting examples of a non-transitory computer-readable medium are nonvolatile memory devices (such as a flash memory device, an erasable programmable read-only memory device, or a mask read-only memory device), volatile memory devices (such as a static random access memory device or a dynamic random access memory device), magnetic storage media (such as an analog or digital magnetic tape or a hard disk drive), and optical storage media (such as a CD, a DVD, or a Blu-ray Disc).

The apparatuses and methods described in this application may be partially or fully implemented by a special purpose computer created by configuring a general purpose computer to execute one or more particular functions embodied in computer programs. Such apparatuses and methods may be described as computerized apparatuses and computerized methods. The functional blocks and flowchart elements described above serve as software specifications, which can be translated into the computer programs by the routine work of a skilled technician or programmer.

The computer programs include processor-executable instructions that are stored on at least one non-transitory computer-readable medium. The computer programs may also include or rely on stored data. The computer programs may encompass a basic input/output system (BIOS) that interacts with hardware of the special purpose computer, device drivers that interact with particular devices of the special purpose computer, one or more operating systems, user applications, background services, background applications, etc.

The computer programs may include: (i) descriptive text to be parsed, such as HTML (hypertext markup language), XML (extensible markup language), or JSON (JavaScript Object Notation), (ii) assembly code, (iii) object code generated from source code by a compiler, (iv) source code for execution by an interpreter, (v) source code for compilation and execution by a just-in-time compiler, etc. As examples only, source code may be written using syntax from languages including C, C++, C#, Objective-C, Swift, Haskell, Go, SQL, R, Lisp, Java®, Fortran, Perl, Pascal, Curl, OCaml, JavaScript®, HTML5 (Hypertext Markup Language 5th revision), Ada, ASP (Active Server Pages), PHP (PHP: Hypertext Preprocessor), Scala, Eiffel, Smalltalk, Erlang, Ruby, Flash®, Visual Basic®, Lua, MATLAB, SIMULINK, and Python®.

What is claimed is:

1. A computer system comprising:
memory hardware configured to store medical appointment data, appointment swap preference data, and computer-executable instructions; and
processor hardware configured to execute the instructions, wherein the instructions include:
receiving, via a first user interface, a medical appointment slot swap request from a first patient, wherein the first user interface is a user interface of a first user device remote from a database of the memory hardware;
accessing a set of swappable medical appointment slots from the database of the memory hardware, wherein each of the set of swappable medical appointment slots comprises a medical appointment slot having an availability status set to booked in the medical appointment data, and a swap possible status set to true in the appointment swap preference data, and accessing the set of swappable medical appointment slots includes identifying a storage location in the database of the memory hardware and executing a read instruction to read electronically stored data from the database at the identified storage location;
electronically transmitting the set of swappable medical appointment slots to the first user device via at least one of a wired communication network and a wireless communication network;
receiving, via the first user interface, a selection of one of the set of swappable medical appointment slots from the first patient;
transmitting a notification to a second user interface corresponding to a second patient associated with the selected one of the set of swappable medical appointment slots;
receiving, via the second user interface, an approval decision input from the second patient, wherein the approval decision input includes a swap acceptance or a swap denial, and the second user interface is a user interface of a second user device remote from the database of the memory hardware; and
in response to receiving the swap acceptance, cancelling a current booked status of the selected one of the set of swappable medical appointment slots associated with the second patient, and assigning a booked status of the selected one of the set of swappable medical appointment slots to the first patient, wherein cancelling the current booked status and assigning the booked status includes modifying at least one electronically stored data entry of the database of the memory hardware to electronically store modified data.

2. The computer system of claim 1, wherein the processor hardware is configured to execute the instructions to, in response to receiving the swap acceptance:
identify an alternative medical appointment slot having an availability status set to available; and
assign a booked status of the alternative medical appointment slot to the second patient.

3. The computer system of claim 1, wherein the processor hardware is configured to execute the instructions to, in response to receiving the swap denial, transmit a notification to the first user interface indicative of the swap denial.

4. The computer system of claim 3, wherein the processor hardware is configured to execute the instructions to, in response to receiving the swap denial:
identify an alternative medical appointment slot having an availability status set to available; and
assign a booked status of the alternative medical appointment slot to the first patient.

5. The computer system of claim 1, wherein the processor hardware is configured to execute the instructions to, in response to receiving the selection of one of the set of swappable medical appointment slots from the first patient:
add the selected one of the set of swappable medical appointment slots to a waitlist; and
assign a high priority value and swap request appointment description to the selected one of the set of swappable medical appointment slots.

6. The computer system of claim 1, wherein adding the selected one of the set of swappable medical appointment slots to a waitlist includes:
setting a request status value associated with the selected one of the set of swappable medical appointment slots to waitlist; and
setting a practitioner status value associated with the selected one of the set of swappable medical appointment slots to action needed.

7. The computer system of claim 1, wherein assigning the booked status includes storing appointment details in the database via an appointment entity application programming interface (API).

8. The computer system of claim 1, wherein the processor hardware is configured to execute the instructions to:
receive a cancellation of a medical appointment slot having an availability status set to booked; and
transmit a notification to the first user interface in response to the first patient having a waitlist request for the medical appointment slot, wherein the notification indicates availability of the medical appointment slot.

9. The computer system of claim 8, wherein the processor hardware is configured to execute the instructions to:
display an acceptance option on the first user interface;
receive an acceptance option input selection from the first patient via the first user interface; and
in response to the acceptance option input selection including a slot acceptance, assign a booked status of the medical appointment slot to the first patient.

10. The computer system of claim 9, wherein the processor hardware is configured to execute the instructions to, in response to the acceptance option input selection including a slot denial, cancel the waitlist request for the medical appointment slot associated with the first patient.

11. A method for medical appointment swap management, the method comprising:
storing medical appointment data and appointment swap preference data in memory hardware;
receiving, via a first user interface, a medical appointment slot swap request from a first patient, wherein the first user interface is a user interface of a first user device remote from a database of the memory hardware;
accessing a set of swappable medical appointment slots from a database of the memory hardware, wherein each of the set of swappable medical appointment slots comprises a medical appointment slot having an availability status set to booked in the medical appointment data, and a swap possible status set to true in the appointment swap preference data, and accessing the set of swappable medical appointment slots includes identifying a storage location in the database of the memory hardware and executing a read instruction to read electronically stored data from the database at the identified storage location;
electronically transmitting the set of swappable medical appointment slots to the first user device via at least one of a wired communication network and a wireless communication network;
receiving, via the first user interface, a selection of one of the set of swappable medical appointment slots from the first patient;
transmitting a notification to a second user interface corresponding to a second patient associated with the selected one of the set of swappable medical appointment slots;
receiving, via the second user interface, an approval decision input from the second patient, wherein the approval decision input includes a swap acceptance or a swap denial, and the second user interface is a user interface of a second user device remote from the database of the memory hardware; and
in response to receiving the swap acceptance, cancelling a current booked status of the selected one of the set of swappable medical appointment slots associated with the second patient, and assigning a booked status of the selected one of the set of swappable medical appointment slots to the first patient, wherein cancelling the current booked status and assigning the booked status includes modifying at least one electronically stored data entry of the database of the memory hardware to electronically store modified data.

12. The method of claim 11, further comprising, in response to receiving the swap acceptance:
identifying an alternative medical appointment slot having an availability status set to available; and
assigning a booked status of the alternative medical appointment slot to the second patient.

13. The method of claim 11, further comprising, in response to receiving the swap denial, transmitting a notification to the first user interface indicative of the swap denial.

14. The method of claim 13, further comprising, in response to receiving the swap denial:
identifying an alternative medical appointment slot having an availability status set to available; and
assigning a booked status of the alternative medical appointment slot to the first patient.

15. The method of claim 11, further comprising, in response to receiving the selection of one of the set of swappable medical appointment slots from the first patient:
adding the selected one of the set of swappable medical appointment slots to a waitlist; and
assigning a high priority value and swap request appointment description to the selected one of the set of swappable medical appointment slots.

16. The method of claim 11, wherein adding the selected one of the set of swappable medical appointment slots to a waitlist includes:
setting a request status value associated with the selected one of the set of swappable medical appointment slots to waitlist; and
setting a practitioner status value associated with the selected one of the set of swappable medical appointment slots to action needed.

17. The method of claim 11, wherein assigning the booked status includes storing appointment details in the database via an appointment entity application programming interface (API).

18. The method of claim 11, further comprising:
receiving a cancellation of a medical appointment slot having an availability status set to booked; and
transmitting a notification to the first user interface in response to the first patient having a waitlist request for the medical appointment slot, wherein the notification indicates availability of the medical appointment slot.

19. The method of claim 18, further comprising:
displaying an acceptance option on the first user interface;
receiving an acceptance option input selection from the first patient via the first user interface; and
in response to the acceptance option input selection including a slot acceptance, assigning a booked status of the medical appointment slot to the first patient.

20. The method of claim 19, further comprising, in response to the acceptance option input selection including a slot denial, cancelling the waitlist request for the medical appointment slot associated with the first patient.
